(19) United States
(12) Patent Application Publication
(10) Pub. No.: US 2008/0081060 A1
(43) Pub. Date: Apr. 3, 2008

(54) CHITOSAN-COATED CALCIUM SULFATE BASED MEDICAMENT DELIVERY SYSTEM

(76) Inventors: Warren O. Haggard, Bartlett, TN (US); Joel D. Bumgardner, Memphis, TN (US); Scott Noel, Memphis, TN (US); Kelly Richelsoph, Memphis, TN (US); Youling Yuan, Memphis, TN (US)

Correspondence Address: W. EDWARD RAMAGE, COMMERCE CENTER SUITE 1000, 211 COMMERCE ST, NASHVILLE, TN, 37201, US

(21) Appl. No.: 11/866,841

(22) Filed: Oct. 3, 2007

Related U.S. Application Data
(60) Provisional application No. 60/849,075, filed on Oct. 3, 2006.

(51) Int. Cl. A61F 13/00; A61K 31/135; A61K 31/35; A61K 38/00; A61P 43/00

(52) U.S. Cl. 424/422; 514/2; 514/459; 514/646

(57) ABSTRACT

A biodegradable medicament delivery system comprising a multi-layered calcium-sulfate based drug delivery vehicle. The vehicle comprises a calcium sulfate center or core with the medicament or medicaments, encased in one or more layers of chitosan. The chitosan may be cross-linked with a cross-linking agent. The vehicle may comprise any suitable shape, including, but not limited to, a sphere, bead or pellet. Medicaments include, but are not limited to, antibiotics, anesthetics, growth factors, and proteins. A physician can implant the coated vehicles into the desired site to create a beneficial, localized treatment that produces high local concentrations of medication while reducing the overall serum concentration throughout the body.

This application claims priority to U.S. Provisional Patent Application No. 60/849,075, filed Oct. 3, 2006, by Warren O. Haggard, et al., and is entitled in whole or in part to that filing date for priority. The entire disclosure, specification and drawings of Provisional Patent Application No. 60/849,075 are incorporated herein in their entireties by reference.

### TECHNICAL FIELD

The present invention relates to a medicament delivery system. More particularly, the present invention relates to a material for use as a vehicle for delivery of medicaments to a graft or wound or defect site.

### BACKGROUND OF THE INVENTION

Localized drug delivery is an emerging area of study aimed at providing an alternative to the conventional methods currently being used by clinicians. Oral and intravenous delivery of drugs has long been the method of treatment to most patients. However, the need exists to develop systems that avoid some of the drawbacks seen with typical delivery methods. In conventional whole-body dosing, high levels of drug must be administered to achieve satisfactory results in eradication of infection or pain. This type of dosing method can lead to systemic toxicity resulting from overdosing. Another concern regarding oral or intravenous drug delivery is that underdosing may occur in order to keep serum drug levels lower. When a patient is underdosed, antibiotic resistance can occur. Antibiotic resistance develops as bacteria become resistant to a certain drug. Resistance is built up in the body due to long term use or underdosing as the bacteria acquire defense mechanisms to the drug and become increasingly harder to eradicate. An optimized local drug delivery system can possibly correct these faults of normal whole-body dosing.

More than two million people in the United States each year suffer bone diseases, defects, or traumatic injuries that require orthopedic implants and/or bone grafting materials. The current gold standard for bone grafting is an autograft because there is no risk of disease transmission or immunological rejection. However, autografts are severely limited in quantity and sometimes quality, and they lead to pain and risk of infection at the donor site. Allografts are also popular. However, with allografts the risk of disease transmission and immunological reactions is present.

Similarly, the number of procedures due to musculoskeletal related injuries and conditions tops 7.5 million every year in the United States. This number is expected to rise with an aging population and an increasing number of sports and automobile accidents. The American Academy of Orthopaedic Surgeons reported that by the year 2020, there will be over 600,000 joint replacement surgeries a year. These projections are another indicator of the need for progressive alternative treatment methods in relation to infection, pain, and restoration in bone defect sites. There are different carrier materials for localized delivery systems. Some systems are delivered via a non-degradable material. Antibiotic-loaded bone cement is recognized as the current gold standard for orthopedic surgeons when treating patients locally with antibiotics. Polymethylmethacrylate (PMMA) is the chemical compound name for bone cement. PMMA beads have been studied as a carrier for antibiotics with successful results in terms of drug elution and inhibition of bacterial activity.

The major issue with bone cement as a carrier vehicle is the additional surgical procedure necessary to remove the bone cement from the patient as it is not degradable. Because the bone cement is not degradable in vivo, there exists the possibility of a foreign body response to the material after it no longer elutes therapeutic levels of antibiotic. Other drawbacks to this system are the possible adhesion of bacteria to the surface of the PMMA and the opportunity for bacterial resistance to be achieved due to sub-therapeutic levels of antibiotic being eluted over a long duration. Although a PMMA delivery system acts as a very useful mechanism in the slow and predictable release of antibiotic, it is not without several faults that make it less than ideal in the treatment of musculoskeletal disease or injury.

Several synthetic materials are currently being used as replacements for bone autografts and allografts. Calcium compounds such as calcium sulfate and calcium phosphate are some of the most commonly used materials. These materials are osteoconductive, but their degradation rate is difficult to control, and they are very brittle. Polymers such as polylactic and polyglycolic acid (PLA/PGA) and their copolymers are also being investigated as bone graft substitutes. However, these materials have been shown to release acidic degradation products that increase inflammation at the implant site and impair healing.

Calcium sulfate in the form of Plaster of Paris has been used for more than one hundred years in the treatment of bone defects and is recognized as an effective bone graft substitute. Calcium sulfate has been shown to act as space filler to help restore bone structure. Calcium sulfate also inhibits the growth of soft tissue, displays osteoconductive properties to aid in bone regeneration, and is very compatible with osteogenic cells. One of the advantages of certain types of calcium sulfate is a uniform absorption rate that can equal the rate of new bone growth. Calcium sulfates are considered a safe bone graft substitute since calcium sulfate avoids issues of contamination with biological viruses and diseases that may be found with allograft tissues. One disadvantage of calcium sulfate pellets is that they can cause excessive wound drainage from their rapid degradation. The wound drainage from dissolved pellets is an ongoing clinical concern with clinical users.

Staphylococcus epidermidis is the one of the most commonly found infectious bacteria in the human body. Staphylococcus epidermidis can cause many forms of infection: superficial skin lesions (boils, styes) and localized abscesses in other sites; and deep-seated infections such as bone osteomyelitis and endocarditis and more serious skin infections (furunculosis). It is a major cause of hospital acquired (nosocomial) infection of surgical wounds and indwelling medical devices.

The local presence of antibiotics like gentamicin, a member of the aminoglycoside family of antibiotics, has the ability to kill a wide variety of bacteria. Gentamicin binds to components in the bacterial cell which result in the production of abnormal proteins. These proteins are necessary for the bacteria's survival. The production of these abnormal proteins is ultimately fatal to the bacteria. Gentamicin is not absorbed from the gut and is therefore only given by injection, infusion or by local delivery system.

Another antibiotic for serious infections is tobramycin. Tobramycin sulfate is an amino glycoside antibiotic used to treat various types of bacterial infections, particularly Gram-negative infections. Tobramycin works by binding to a site on the bacterial ribosome and causing the genetic code to be misread. Like all amino glycosides, tobramycin does not pass the gastrointestinal tract. For systemic use of tobramycin, the delivery can only be given by intravenous and intramuscular injection and by local delivery system.

Daptomycin is another antibiotic that can be given by intravenously or intramuscularly but not orally. Local delivery of daptomycin has not been extensively researched. Daptomycin is a lipopeptide antibiotic. It is active only against Gram-positive organisms. It is a true antibiotic in that it is a naturally occurring compound which is found in the soil saprotroph, Streptomyces roseosporus. The compound was initially called LY146032 and was first discovered by Eli Lilly in the 1980s as part of their drug development program. The rights to LY146032 were bought by Cubist Pharmaceuticals in 1997, who brought it to the US market in November 2003 as Cubicin®. It has proven in vitro activity against Enterococci (including glycopeptide-resistant Enterococci [GRE]), Staphylococci 3 (including methicillin-resistant Staphylococcus aureus), Streptococci, and Corynebacteria.

The current approach to control pain in graft/wound/defect sites is to administer intravenous or oral medication. Treatment of many musculoskeletal infections can be improved by local delivery of the antibiotics like gentamicin and tobramycin. The localized delivery of antibiotics has emerged as a progressive alternative for treatment of infected bone defects. By administering antibiotics locally instead of orally or intravenously, high concentrations of the drug can be reached with low serum concentrations. Previous local delivery studies have demonstrated that an antibiotic can be released over a prolonged period of time, although the majority of the release occurs within 24 hours. If this burst of antibiotics in the first 24 hours can be modified to increase drug delivery levels over the following days, a more effective treatment of the infections can be developed.

The antibiotic(s) released from the local delivery systems should satisfy certain criteria. First, the released antibiotic should be active against the most common bacterial pathogens involved in infections. Second, maintaining the antibiotic concentration above the Minimum Inhibitory Concentration (MIC) levels is critical in treating bacterial bone infection. The locally released antibiotic concentrations should exceed several times (usually 10 times) the minimum inhibitory concentration (MIC) for the involved pathogen. Third, the antibiotic concentration should not provoke any adverse effects and exhibit low systemic concentration. Fourth, the antibiotic should be stable at body temperature and also hydrophilic to ensure proper diffusion from the carrier.

As drug delivery systems are used in localized treatment applications for bone infections, a clinical need to extend the elution of therapeutic agents for longer treatment periods is being sought. The localized delivery of antibiotics has emerged as an alternative to conventional methods of treatment for certain bone defects. The local delivery of anesthetics to provide pain management after orthopedic procedures would expand clinical treatment options for the orthopedic surgeons. The site where autograft bone tissue is harvested from becomes very painful after surgery. The delivery of localized anesthetic will help to alleviate this pain while providing osteogenic behavior. As with antibiotics, by administering anesthetics locally instead of orally or intravenously, higher concentrations of the therapeutic agent can be attained and maintained with low serum concentrations. The undesirable effects associated with anesthetics at high serum concentrations can be avoided with a local delivery system.

Many materials have been investigated as vehicles to deliver therapeutic agents such as growth factors, antibiotics, or anesthetics to graft or implant sites. However, these biological compounds do not bind well to many of these materials, and because the degradation rate is difficult to control, the growth factors or other compounds are often released too quickly or not at a biologically driven rate.

Accordingly, what is needed is a local drug delivery material that overcomes the problems associated with other such materials, particularly with a controllable degradation rate, the ability to bind biological compounds well, appropriate pore sizes, good interconnected porosity, and mechanical properties sufficient to support bone during healing.

### SUMMARY OF THE INVENTION

This invention is directed to a biodegradable medicament delivery system comprising a multi-layered calcium-sulfate based drug delivery vehicle. In one exemplary embodiment, the vehicle comprises a calcium sulfate center or core containing the medicament or medicaments, encased in one or more layers of chitosan. The chitosan may be cross-linked with a cross-linking agent. In one exemplary embodiment, the cross-linking agent is genipin.

The vehicle may comprise any suitable shape, including, but not limited to, a sphere, bead or pellet. Medicaments include, but are not limited to, antibiotics, anesthetics, growth factors, and proteins. A physician can implant the coated vehicles into the desired site to create a beneficial, localized treatment that produces high local concentrations of medication while reducing the overall serum concentration throughout the body.

### BRIEF DESCRIPTION OF THE DRAWINGS

FIG. 1 shows a idealized cross-section of a calcium sulfate drug delivery vehicle in the shape of a spherical bead with multiple chitosan layers, in accordance with an exemplary embodiment of the present invention.

FIG. 2 shows the chemical structure of chitin and chitosan monomeric units.

FIG. 3 shows the chemical structure of genipin, and the cross-linking of genipin with chitosan.

FIG. 4 shows dissolution profiles of several variations of uncoated and chitosan-coated calcium sulfate pellets loaded with gentamicin, in accordance with an exemplary embodiment of the present invention.

FIG. 5 shows dissolution profiles of several variations of uncoated and chitosan-coated calcium sulfate pellets loaded with tobramycin, in accordance with an exemplary embodiment of the present invention.

FIG. 6 shows dissolution profiles of several variations of uncoated and chitosan-coated calcium sulfate pellets loaded with gentamicin, in accordance with an exemplary embodiment of the present invention.

FIG. 7 shows dissolution profiles of several variations of uncoated and chitosan-coated calcium sulfate pellets loaded with tobramycin, in accordance with an exemplary embodiment of the present invention.

FIG. 8 shows dissolution profiles of several variations of uncoated and chitosan-coated calcium sulfate pellets loaded with gentamicin, in accordance with an exemplary embodiment of the present invention.

FIG. 9 shows dissolution profiles of several variations of uncoated and chitosan-coated calcium sulfate pellets loaded with tobramycin, in accordance with an exemplary embodiment of the present invention.

FIG. 10 shows dissolution profiles of several variations of uncoated and chitosan-coated calcium sulfate pellets loaded with tobramycin, in accordance with an exemplary embodiment of the present invention.

FIG. 11 shows dissolution profiles of several variations of uncoated and cross-linked chitosan-coated calcium sulfate pellets loaded with gentamicin, in accordance with an exemplary embodiment of the present invention.

FIG. 12 shows dissolution profiles of several variations of uncoated and cross-linked chitosan-coated calcium sulfate pellets loaded with tobramycin, in accordance with an exemplary embodiment of the present invention.

FIG. 13 shows dissolution profiles of several variations of uncoated and chitosan-coated calcium sulfate pellets loaded with lidocaine, in accordance with an exemplary embodiment of the present invention.

FIG. 14 shows elution profiles of several variations of uncoated and chitosan-coated calcium sulfate pellets loaded with gentamicin, in accordance with an exemplary embodiment of the present invention.

FIG. 15 shows elution profiles of several variations of uncoated and chitosan-coated calcium sulfate pellets loaded with tobramycin, in accordance with an exemplary embodiment of the present invention.

FIG. 16 shows elution profiles of several variations of uncoated and chitosan-coated calcium sulfate pellets loaded with gentamicin, in accordance with an exemplary embodiment of the present invention.

FIG. 17 shows elution profiles of several variations of uncoated and chitosan-coated calcium sulfate pellets loaded with tobramycin, in accordance with an exemplary embodiment of the present invention.

FIG. 18 shows elution profiles of several variations of uncoated and chitosan-coated calcium sulfate pellets loaded with gentamicin, in accordance with an exemplary embodiment of the present invention.

FIG. 19 shows elution profiles of several variations of uncoated and chitosan-coated calcium sulfate pellets loaded with tobramycin, in accordance with an exemplary embodiment of the present invention.

FIG. 20 shows elution profiles of several variations of uncoated and cross-linked chitosan-coated calcium sulfate pellets loaded with gentamicin, in accordance with an exemplary embodiment of the present invention.

FIG. 21 shows elution profiles of several variations of uncoated and cross-linked chitosan-coated calcium sulfate pellets loaded with tobramycin, in accordance with an exemplary embodiment of the present invention.

FIG. 22 shows a representative SEM image of a chitosan-coated delivery vehicle in cylindrical pellet form showing cracking.

FIG. 23 shows another representative SEM image of a chitosan-coated delivery vehicle in cylindrical pellet form showing cracking.

FIG. 24 shows another representative SEM image of a chitosan-coated delivery vehicle in cylindrical pellet form showing cracking.

### DETAILED DESCRIPTION OF EXEMPLARY EMBODIMENTS

In one exemplary embodiment, the invention described herein is a novel biodegradable medicament delivery system. An alternative approach to controlling pain and infection in a graft or wound or defect site through the administration of intravenous or oral medication, the present invention uses biocompatible and resorbable coated products as carrier vehicles for a medicament. Medicaments include, but are not limited to, antibiotics, anesthetics, growth factors, and proteins. A physician can implant the coated vehicles into the site to create a beneficial, localized treatment that produces high local concentrations of medication while reducing the overall serum concentration throughout the body.

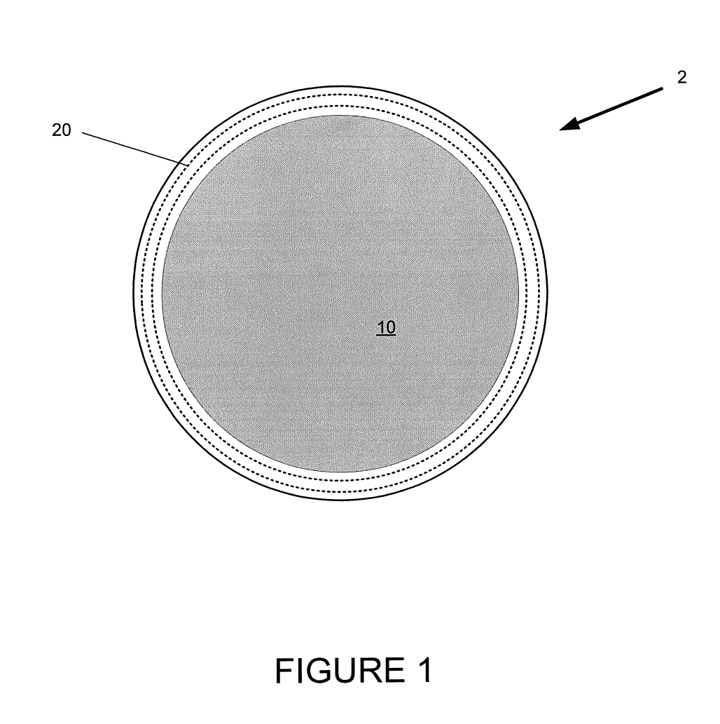

FIG. 1 shows a cross-section of an exemplary embodiment of a coated carrier vehicle in the shape of a bead. The vehicle 2 comprises a calcium sulfate center 10 coated with one or more layers of chitosan 20. Calcium sulfate carrier vehicles may be fabricated as beads or pellets, or some other suitable shape, such as small cylinders. Calcium sulfate is widely used as a bone graft substitute and therapeutic agent delivery system. It is a biodegradable delivery system that has been used successfully as a local delivery system for several different antibiotics. The calcium sulfate center 10 comprises a mixture of calcium sulfate with various medicaments, including antibiotics such as, but not limited to, gentamicin, tobramycin, or daptomycin, or anesthetics including, but not limited to, lidocaine hydrochloride.

Chitosan has a growing presence as a localized drug delivery system. Both of these materials display osteoconductive, biodegradation and carrier compatibility properties that are very useful for the orthopedic application of local medicaments.

Chitosan is a natural polymer that is biodegradable at a controlled rate dependent on its molecular weight and degree of deacetylation. It is non-toxic and biocompatible. It also has been shown to have some antibacterial, antifungal, and osteogenic properties, and both it and its degradation products enhance wound healing. Chitosan has bioadhesive characteristics and readily binds to negatively charged surfaces such as mucosal membranes. Chitosan enhances the transport of polar drugs across epithelial surfaces, and is biocompatible and biodegradable. In addition, chitosan can effectively accumulate and retain biologically active molecules and promote controlled release of those molecules due to its pH-dependent cationic nature. Chitosan's physical and material characteristics, like its degradation rate, can be modified by cross linking it with other substances.

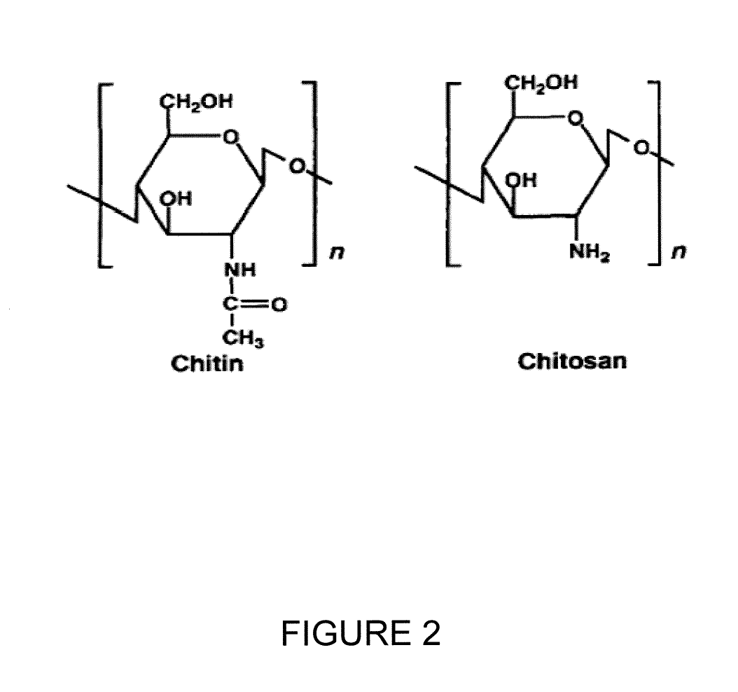

As seen in FIG. 2, chitosan is a linear polysaccharide co-polymer of N-acetyl-glucosamine and N-glucosamine units. Either an acetamido group (—NH—COCH₃) or an amino group (—NH₂) is attached to the C-2 carbon of the glucopyran ring. The degree of deacetylation (DDA) represents the percentage of amino groups attached to the polymer glucopyran rings. When more than 50% of the C-2 attachment is an amino group, i.e. >50% DDA, the material is termed chitosan. When more than 50% of the C-2 attachment is the acetomido group, i.e. >50% acetylated, the material is termed chitin.

The chitosan layer 20 may comprise one or more sub-layers of chitosan. In one exemplary embodiment, there are a total of five layers of chitosan, although there may be more or fewer. In addition, the chitosan may be cross-linked with a cross-linking agent to increase the chitosan's resistance to degradation and to decrease the initial burst effect seen with current methods of localized drug delivery. This results in an extended release profile which enhances the overall performance of the drug delivery system.

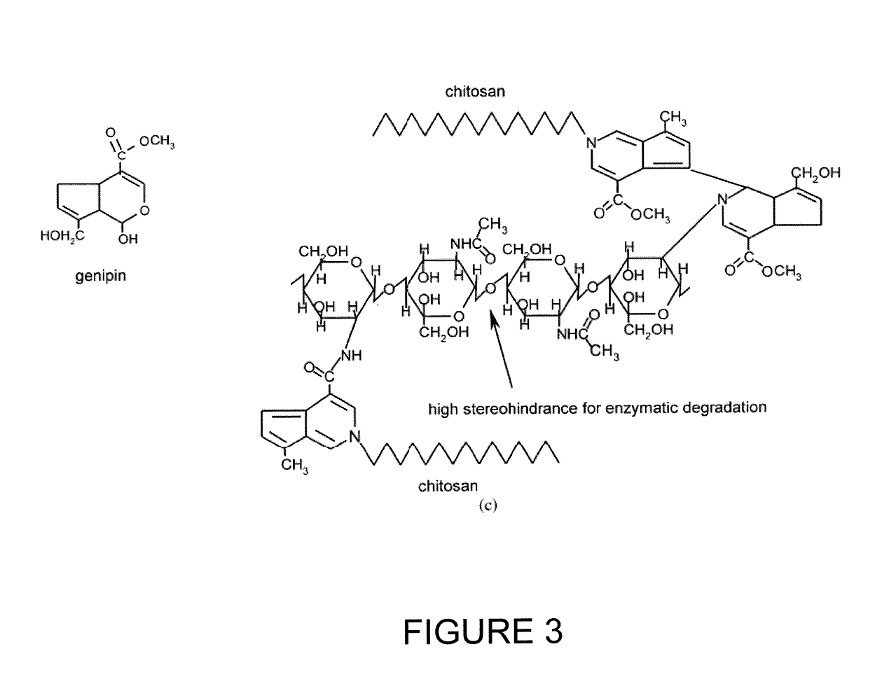

Any appropriate cross-linking agent may be used. In one exemplary embodiment, the cross-linking agent is genipin. The chemical structure of genipin, and the cross-linking of genipin with chitosan, is shown in FIG. 3. Genipin is a naturally-occurring agent with low cytotoxicity.

In one exemplary embodiment, the general steps for creating a cross-linked drug delivery system in accordance with the present invention comprises:

1. Creating a core or center comprising calcium sulfate and one or more medicaments.

2. Coating the center with one or more layers of chitosan.

3. Cross-linking the chitosan layer with a cross-linking agent.

Below are examples of methods of accomplishing these steps to create various forms of the present invention.

### EXAMPLE 1

### Preparation of CaSO₄ Pellets with Gentamicin or Tobramycin

Pellets (or beads) were prepared using 50.0 g of alpha hemihydrate calcium sulfate and 2.6 g of gentamicin sulfate or tobramycin sulfate to make 4.0% by weight antibiotic-loaded pellets. A solution was prepared by mixing 2.6 g of the antibiotic to be loaded with 12.5 g of DI water. This solution was poured over calcium sulfate powder, and then thoroughly mixed with spatula until a free flowing paste was obtained. The paste obtained was poured on a silicon elastomer mold, containing 100 pellet shaped cavities, for casting of the pellets. (A spherical bead mold may be used to create beads.) The paste in pellet mold dries in approximately 10-15 minutes enough for removal of the individual pellets from the mold. The pellets were removed by flexing the mold. These pellets were placed in an oven for 5 to 7 hrs at a temperature of approximately 37° C. to complete the drying.

### EXAMPLE 2

### Preparation of CaSO₄ Pellets with Daptomycin

Pellets with 4.0% by weight daptomycin were made using a potassium sulfate (K₂SO₄) solution instead of DI water. Varying percentages (e.g., 1, 2, 3, 4, and 5 weight percentages) of K₂SO₄ solutions may be used. Potassium sulfate acts as an accelerator and lessens the setting time due to the formation of a compound named syngenite. A ratio of 31.0 ml K₂SO₄ solution to 100 g of calcium sulfate hemihydrate was combined. Daptomycin is added at 2 minutes after mixing. This solution was then thoroughly mixed with spatula until a free flowing paste was obtained. The paste was cast immediately after thorough mixing of the daptomycin. The paste was poured on a silicon elastomer mold, containing 100 pellet-shaped cavities, for casting of the pellets, and subsequent drying, in the same manner as described in Example 1.

The beads or pellets of the above examples may then be coated with chitosan. The chitosan may be cross-linked with a cross-linking agent. By cross-linking the chitosan, the release rate of the medicament is slowed, and release is extended for a longer period of time.

### EXAMPLE 3

### Unlinked Chitosan Coating

A 2.0 weight % chitosan solution is prepared by mixing 1.0 g of chitosan and 49.0 ml of 1.0 wt % acetic acid solution in a glass beaker, stirring for 12 hrs. The CaSO₄-drug loaded pellets were submerged into the chitosan solution (of 87.4% or 92.3% DDA). The coated pellets are placed on polytetrafluoroethylene mesh for drying. A heat gun at 34° C. is moved a circular pattern above the pellets for three minutes. The pellets are turned over and the opposite side was dried with the heat gun for two additional minutes. The coated pellets are then placed in a convection oven for about 1 hour at approximately 37° C. to complete the drying. After one hour, the pellets were removed.

Additional chitosan layers may be added by re-submerging and drying in the same manner as above. The number of chitosan layers may vary. In one exemplary embodiment, five chitosan layers were added. The thickness of the total coating may vary, but in general, the chitosan coatings produced by the above methods was 20-50 microns.

The weight percentage of the chitosan solution may be varied. For example, a 2.5 wt % chitosan solution was obtained by mixing 1.25 g of 87.4% DDA chitosan and 48.75 ml of 1.0% acetic acid solution. A 3.0 wt % chitosan solution was obtained by mixing 1.5 g of 87.4% DDA chitosan and 48.5 ml of 1.0% acetic acid solution.

### EXAMPLE 4

### Cross-Linked Chitosan Coating

The above chitosan-coated pellets may be used with the chitosan layer being cross-linked or unlinked. If the cross-linking is desired, in one exemplary embodiment, the chitosan-coated CaSO₄ pellet may be cross-linked with a genipin solution. Genipin (Molecular Weight=226.23) is one of the most commonly used, naturally occurring cross linking agent, and is a biodegradable molecule with low cytotoxicity. Significantly, genipin has been shown to improve the mechanical properties of chitosan films and coatings which help obtain a better release of antibiotics from pellets with chitosan films or coatings.

A 0.5 wt % genipin solution was prepared by mixing 0.005 g of genipin with 3.0 ml of DI water. The solution was continuously stirred for 30 minutes. The genipin solution was then poured in the 2.0 wt % chitosan solution (87.4% or 92.3% DDA) and stirred for two different time intervals at 2 hours and 8 hours. The color of the solution turned to slight blue using the 2 hour time interval and dark blue using the 8 hour time interval. To accomplish the cross-linking, the chitosan-coated CaSO₄— medicament pellets are then directly placed in the genipin solutions. Time of submergence is one hour (for 5.0 mM solution) or four hours (for 2.0 mM solution). After the cross-linking time was completed, the pellets are removed and dried in a convection oven. Genipin cross-linked chitosan coatings in this embodiment are blue in color. Cross-linked chitosan coatings in this embodiment are more elastic, decrease degradation, and have better mechanical properties than non-crosslinked chitosan coating. Genipin crosslinking produces a chitosan network that is insoluble in acidic and alkaline solutions, but is capable of swelling in aqueous media.

Various chitosan solutions ranging from 1.0 to 3.5 wt % may be used, depending on the handling characteristics desired. A solution of approximately 3.0 wt % chitosan dissolved in approximately 1 wt % acetic acid provides a good viscosity which leads to enhanced adherence to the surface of the calcium sulfate center while undergoing the drying process. A higher viscosity chitosan is less apt to shift during the convective drying process, which allows for multiple layers to be applied to the calcium sulfate in a reasonable timeframe. A weight percentage of chitosan solution higher than 3.5 wt % tends to create a high viscosity solution which inhibits the chitosan from dissolving fully. Lower weight percentages of chitosan result in lower viscosity solutions, which promote dissolution. However, lower viscosity of the solution results in less adhesion to the calcium sulfate center.

In one exemplary embodiment, the thickness of the chitosan layers created was measured to be approximately 34±3.3 μm, although a wider range of thicknesses also can be achieved.

Variations in the above-described methods may be used. In one exemplary embodiment, a reduction in the time between each chitosan coating to 3 to 5 minutes may be used. This faster coating process helps in obtaining more uniform and thick layered coatings on the calcium sulfate pellets. In addition, the method of drying the coated pellets may also be modified to make the coatings more uniform and stable. The use of a hot air source and changes in the temperature and blow rate helped in obtaining a more consistent, stable and uniformly thick coating on the pellets.

Experimental results confirm that a cross-linked chitosan coating on calcium sulfate antibiotic pellets loaded with antibiotics (including, but not limited to, gentamicin, tobramycin or daptomycin) or anesthetics improves the dissolution and elution profile of the antibiotics or anesthetics released from the pellets, and potentially lessens a drainage issue associated with plain or unlinked antibiotic-loaded calcium sulfate pellets. In addition, the released antibiotics from the chitosan and cross-linked chitosan coated calcium sulfate pellets were active against bacteria.

Degradation of the calcium sulfate center or chitosan layer is measured by dissolution testing. Dissolution tests as described below were conducted on coated and uncoated calcium sulfate pellets to determine the effectiveness of the methods described herein. The test monitors the weight change in the samples when exposed to aqueous solutions over a period of time. Samples were weighed before testing. After the initial weight was recorded, samples were placed into a container filled with 20 mL distilled water. These containers were then stored in an agitated water bath with a constant temperature of 37° C. At the first time point of 24 hours, the samples were removed from the containers and placed into a small, plastic weigh boat. The weigh boats were put into a 37° C. convection oven and allowed to dry for a period of one hour. The containers were washed with soap and water and then filled with 20 mL of fresh distilled water. When the drying cycle had concluded, the samples were weighed and this weight was recorded. The calcium sulfate samples were placed back into the containers of fresh distilled water and into the water bath. Samples were subjected to dissolution testing for a minimum period of five days with individual timepoints occurring every 24 hours. When all measurements had been taken, the data was quantified by comparing the individual weights with the initial weights and generating a “percentage of initial weight” profile.

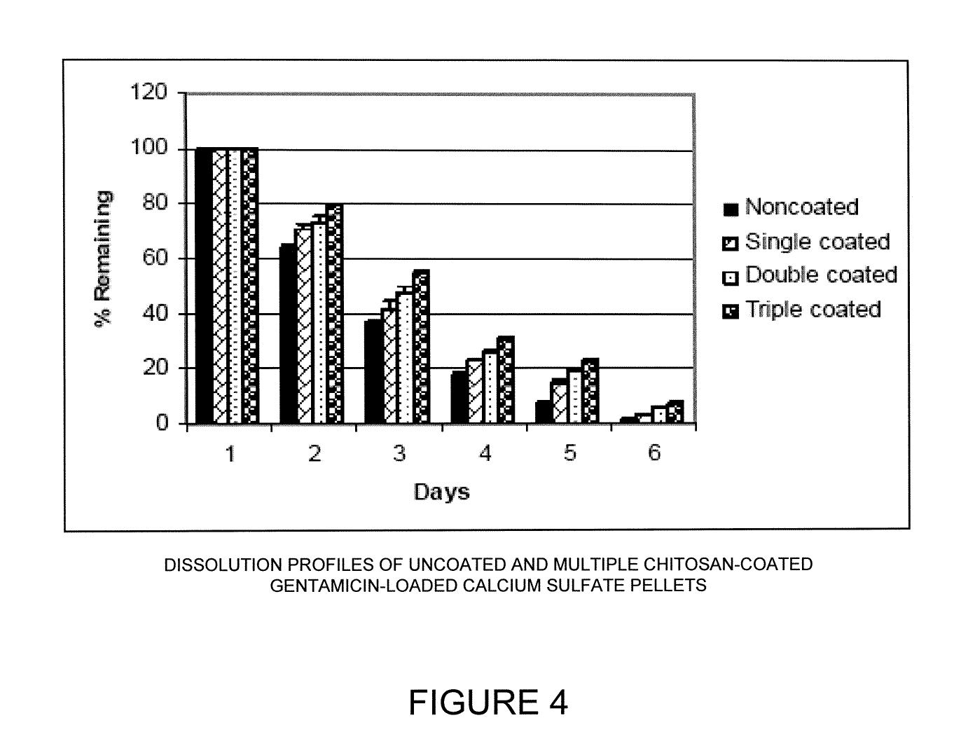

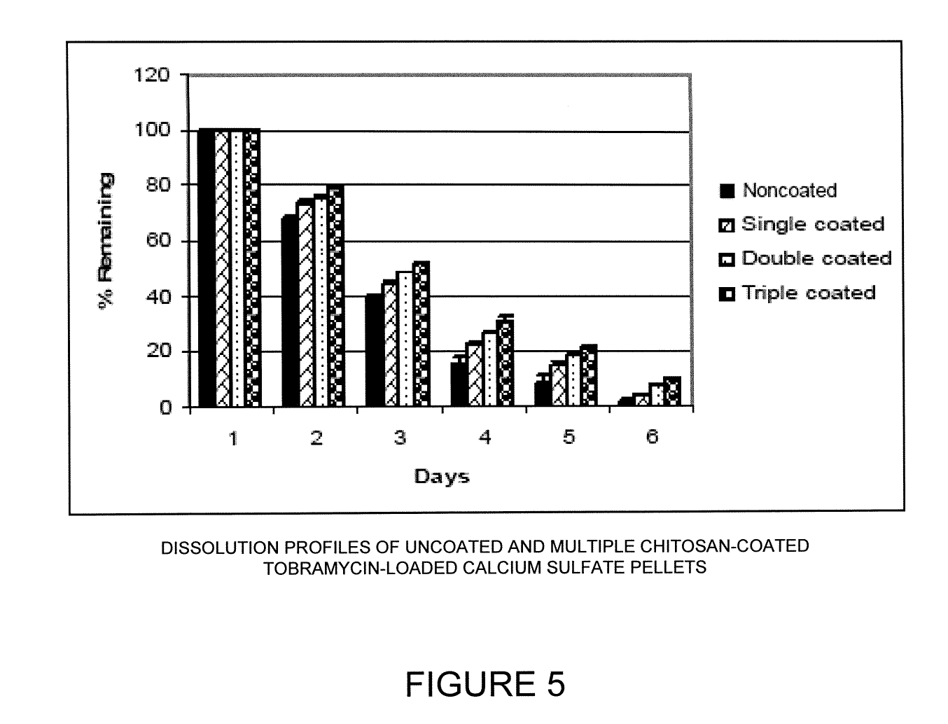

FIG. 4 shows the dissolution profiles for four variations of gentamicin-loaded calcium sulfate pellets (uncoated; single-layer chitosan coated; double-layer chitosan coated; triple-layer chitosan coated). The multiple-layer chitosan-coated pellets showed a higher percentage of the pellet remaining as compared to the uncoated pellets, with more layers corresponding to greater percentage remaining. After five days, the residual weight of uncoated pellets was 10.0%, whereas the residual weight for triple-coated pellets was 22.0%. The dissolution profiles for corresponding tobramycin-loaded calcium sulfate pellets are shown in FIG. 5.

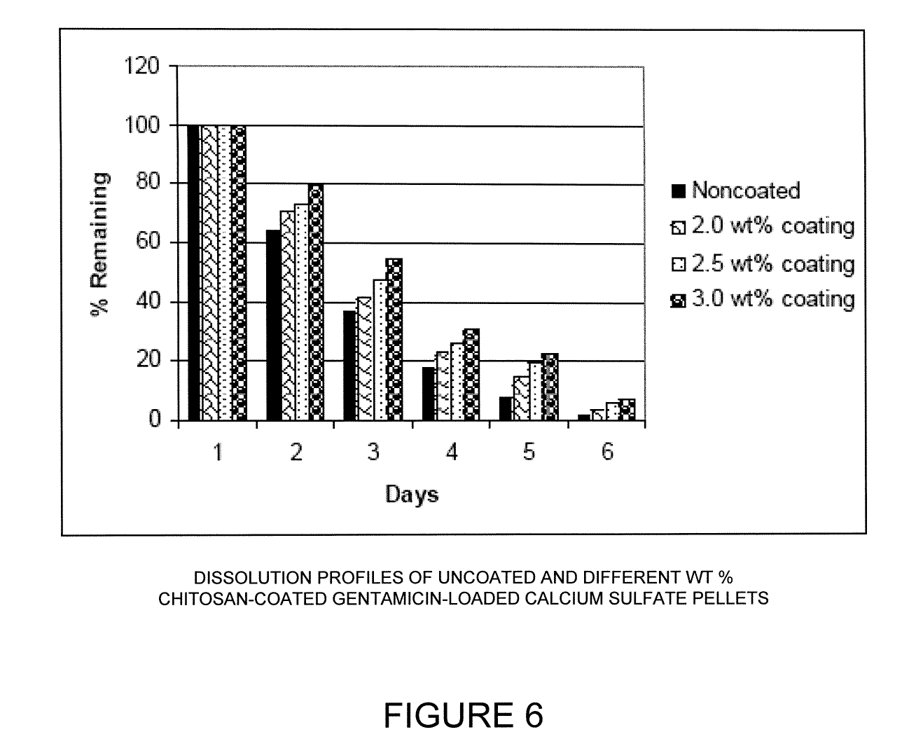

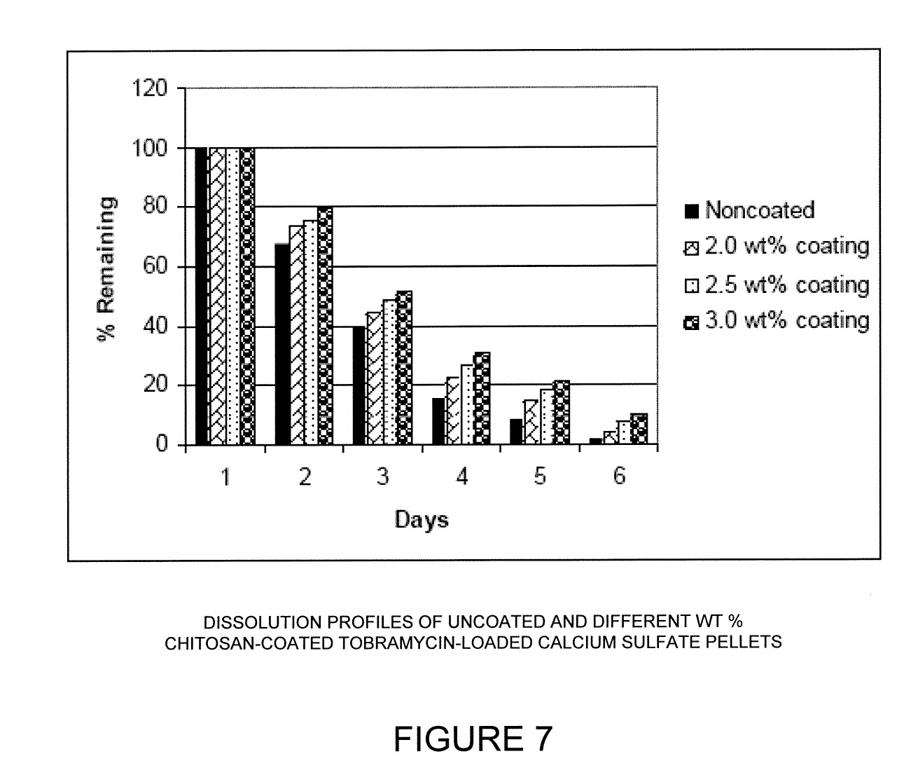

FIG. 6 shows the dissolution profiles for four variations of gentamicin-loaded calcium sulfate pellets (uncoated; 2.0 wt % chitosan; 2.5 wt % chitosan; 3.0 wt % chitosan). The pellets coated with chitosan showed a higher percentage of pellet remaining as compared to the uncoated pellets. The dissolution profiles for corresponding tobramycin-loaded calcium sulfate pellets are shown in FIG. 7.

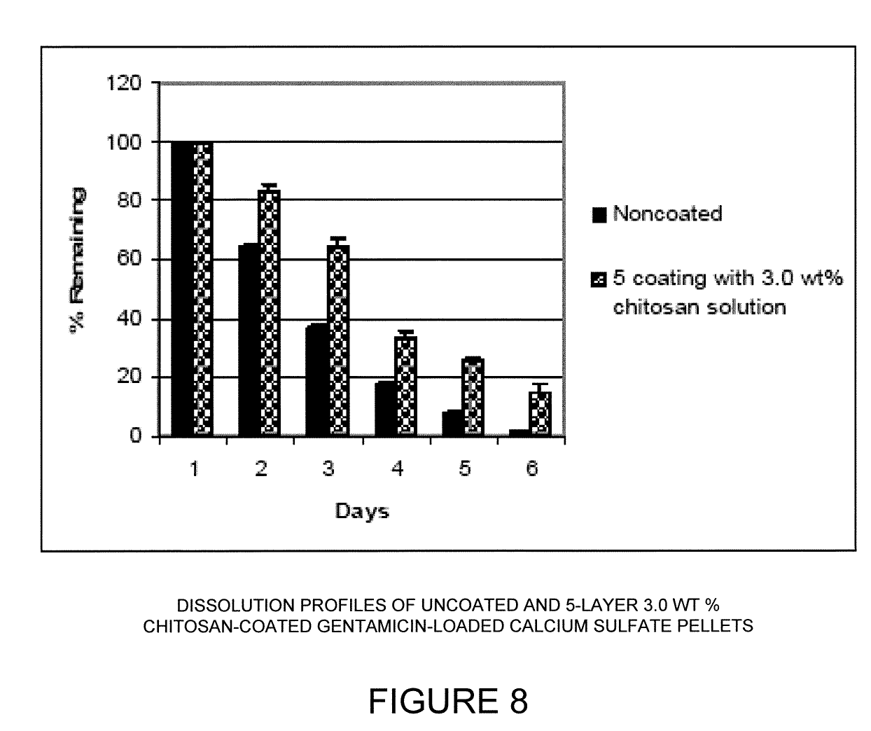

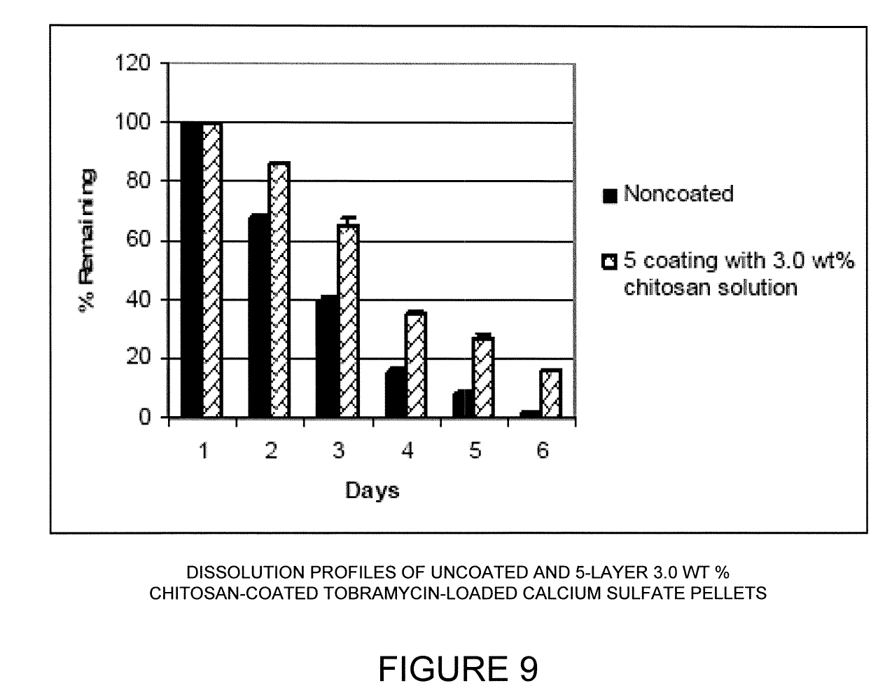

FIG. 8 shows the dissolution profiles for two variations of gentamicin-loaded calcium sulfate pellets (uncoated vs. five-layer chitosan coated with 3.0 wt %). After five days, the residual weight for the uncoated pellets was 10.0%, whereas the pellets with 5-layer coating at 3.0 wt % were at 24.0% residual weight. The dissolution profiles for corresponding tobramycin-loaded calcium sulfate pellets are shown in FIG. 9.

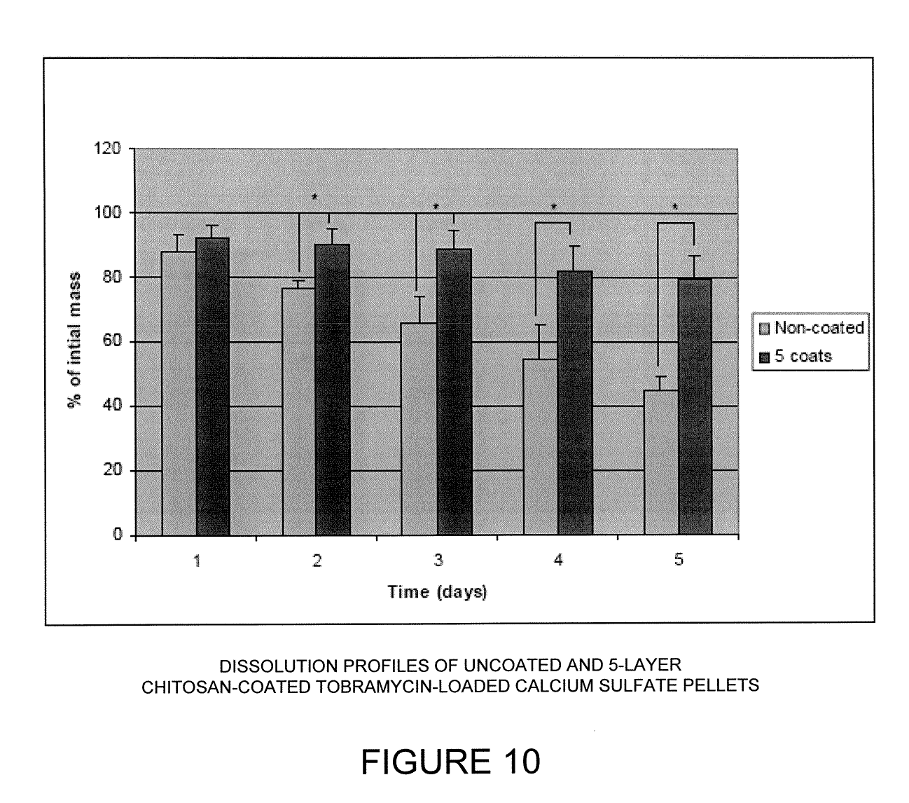

FIG. 10 shows the dissolution profiles for two alternative variations of tobramycin-loaded calcium sulfate pellets (uncoated vs. five-layer chitosan coated). The coated group degraded at a much slower rate than the non-coated group. After five days, the coated group still retained 79.42±7.31% of its initial weight. In comparison, the uncoated group retained only 44.77±4.26% of its initial weight.

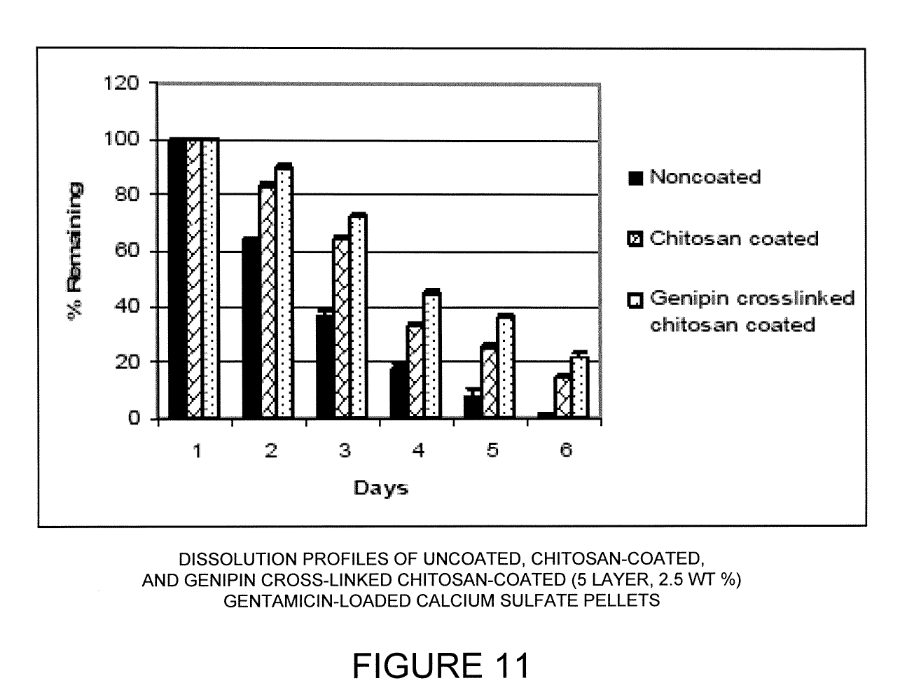

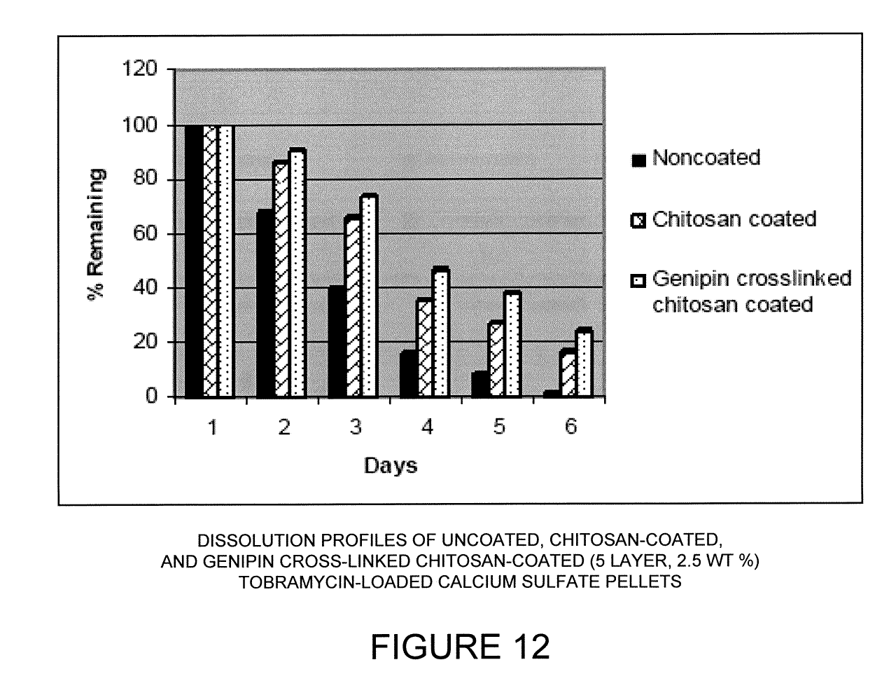

FIG. 11 demonstrates the effect of cross-linking the chitosan coating (2.5 wt %). It shows the dissolution profiles for three variations of gentamicin-loaded calcium sulfate pellets (uncoated; plain chitosan coated; cross-linked chitosan coated). The chitosan coatings were 5 layers. The cross-linked pellets showed the highest percentage of remaining pellet at each time period. After five days, the residual weight for the non-coated pellet sample was 10.0% whereas the pellets with cross-linked chitosan were at 38.0%. The dissolution profiles for corresponding tobramycin-loaded calcium sulfate pellets are shown in FIG. 12.

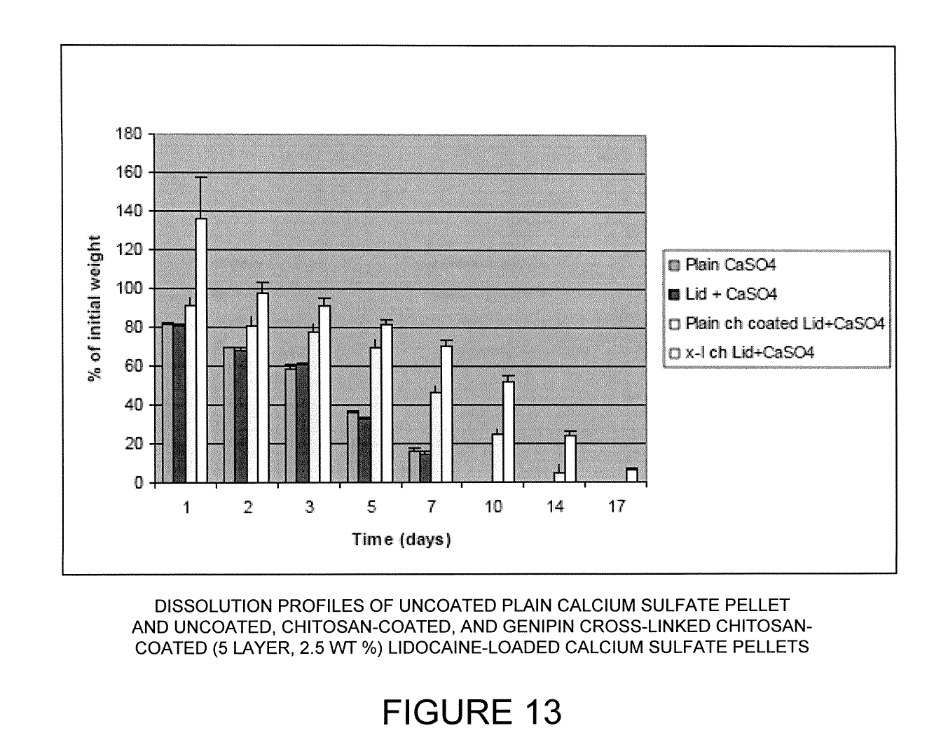

FIG. 13 shows the dissolution profiles for four different variations of pellets (uncoated CaSO₄; uncoated lidocaine-loaded CaSO₄; uncross-linked chitosan coated lidocaine-loaded CaSO₄; cross-linked chitosan coated lidocaine-loaded CaSO₄). The chitosan coating is five layers, when present. The two uncoated groups dissolved completely after day 7, whereas the uncross-linked chitosan coated beads retained 46.0±2.76% and the cross-linked chitosan beads retained 70.45±3.22% of initial weight. The uncross-linked chitosan coated beads dissolved by day 14. The cross-linked chitosan coated beads did not completely dissolve until day 17. These results clearly show that the chitosan layer successfully acted to slow the degradation rate of calcium sulfate, and was even more successful when the chitosan layer was cross-linked.

Monitoring of the drug or medicament release and a representation of the release profile can be obtained with elution testing. Elution tests as described below were conducted on coated and uncoated calcium sulfate pellets to determine the effectiveness of the methods described herein. Groups of five pellets from each particular set of samples to be tested were placed into a 125 mL plastic container. These containers were filled with 20 mL of fresh 1× Phosphate Buffered Saline (PBS) solution (Fisher Scientific, 10× PBS, pH 7.4±0.01, BP399-1). The pH of the PBS was checked before use and determined to be approximately 7.4. The containers were then placed into a 37° C. agitated water bath. At designated timepoints of 1, 3, 6, 24, 48, 96, 168, 240, 336 hours, an aliquot of 1 mL was extracted from each container and placed in a small polypropylene micro-centrifuge tube. These eluates were stored in a typical laboratory freezer (−20° C.) until concentration testing occurred. Once the sample had been pulled, the containers were washed with soap and water and then rinsed twice with distilled water before being filled with 20 mL of fresh PBS. The calcium sulfate pellets were placed back into the container and moved into the water bath until the next timepoint. This process was repeated for various timepoints up to seventeen days.

For the bead-shaped calcium sulfate samples, the volume of PBS was changed for the elution tests. A weight to volume ratio (material to PBS) was used. The amount of PBS used for bead elution studies was 13.75 mL for one bead. The eluates were extracted and stored in the same manner as previously described.

After aliquots had been extracted for all samples at all timepoints, quantification of the eluates was done using two different techniques. Fluorescent polarization immunoassay testing using a TDxFLx device (Abbott Labs) was performed for monitoring antibiotic release. Enzyme-linked immunosorbent assay (ELISA) was the technique used to quantify lidocaine elution.

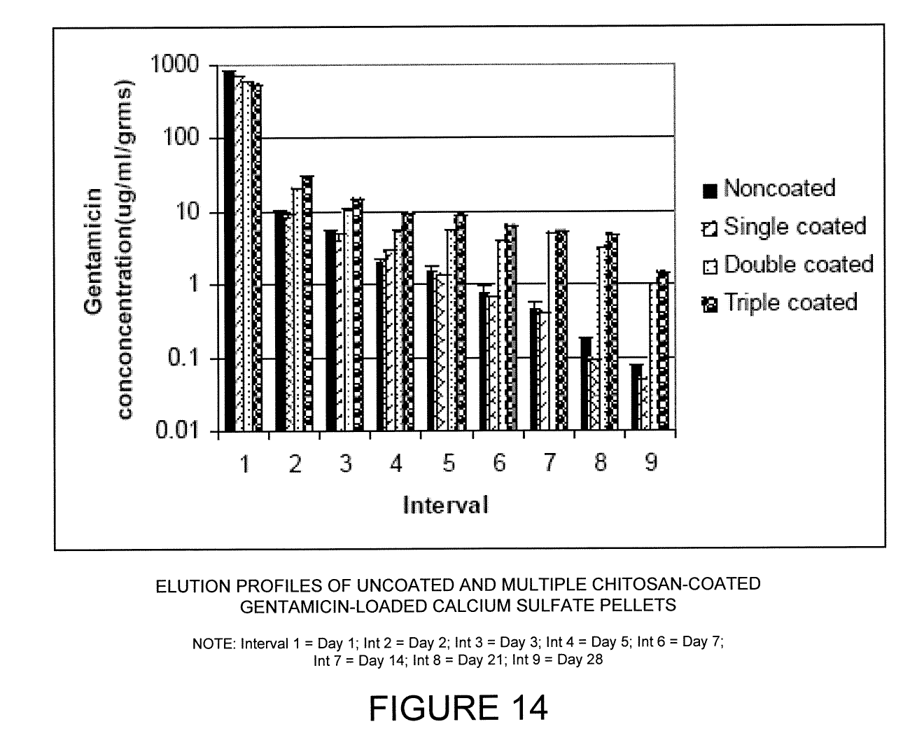

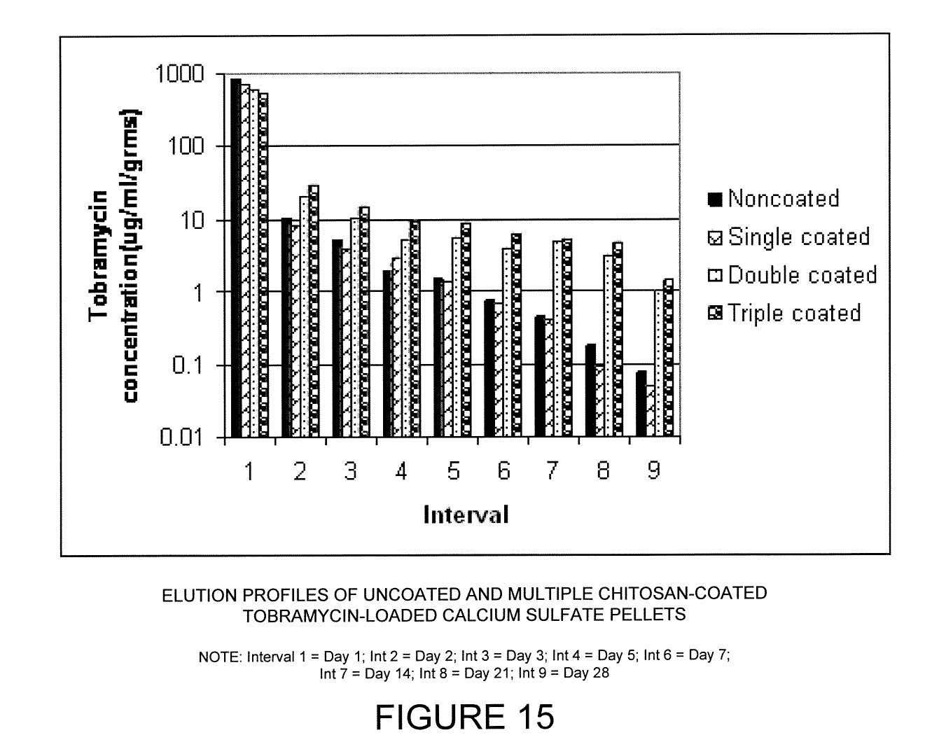

FIG. 14 shows the elution profiles for four variations of gentamicin-loaded calcium sulfate pellets (uncoated; single-layer chitosan coated; double-layer chitosan coated; triple-layer chitosan coated). Time intervals are not linear. The gentamicin concentration has been normalized to the initial mass of the pellets. Peak concentrations of 852 to 525 μg/ml/g occurred on day 1. In general, elution rates stayed higher for multiple-coated pellets. The elution profiles for corresponding tobramycin-loaded calcium sulfate pellets are shown in FIG. 15.

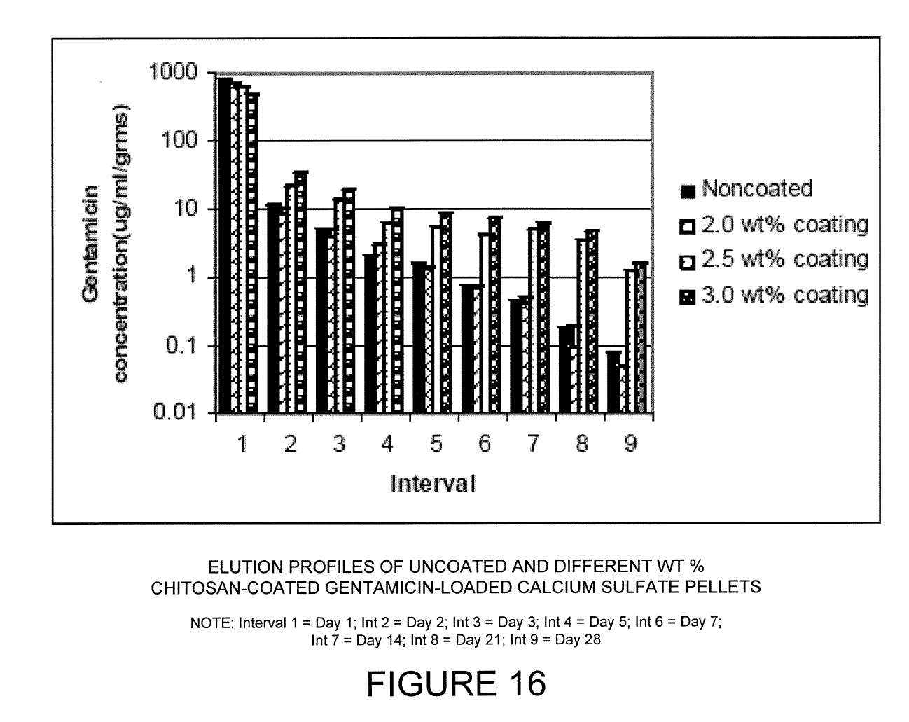

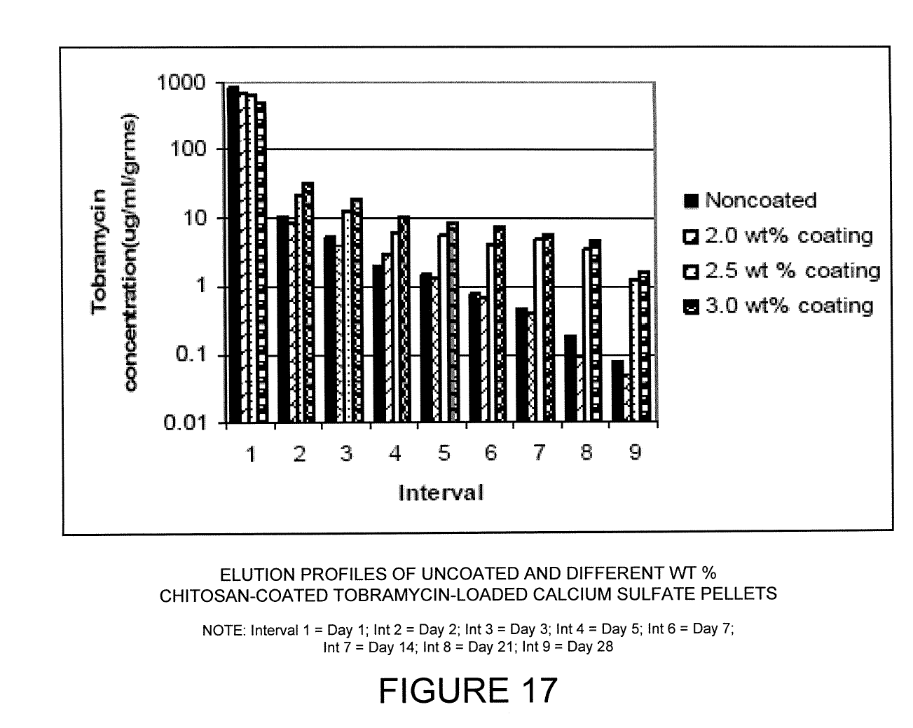

FIG. 16 shows the elution profiles for four variations of gentamicin-loaded calcium sulfate pellets (uncoated; 2.0 wt % chitosan; 2.5 wt % chitosan; 3.0 wt % chitosan). Time intervals are not linear. Peak concentrations of 852 to 512 μg/ml/g occurred on day 1. In general, elution rates stayed higher for multiple-coated pellets. The elution profiles for corresponding tobramycin-loaded calcium sulfate pellets are shown in FIG. 17.

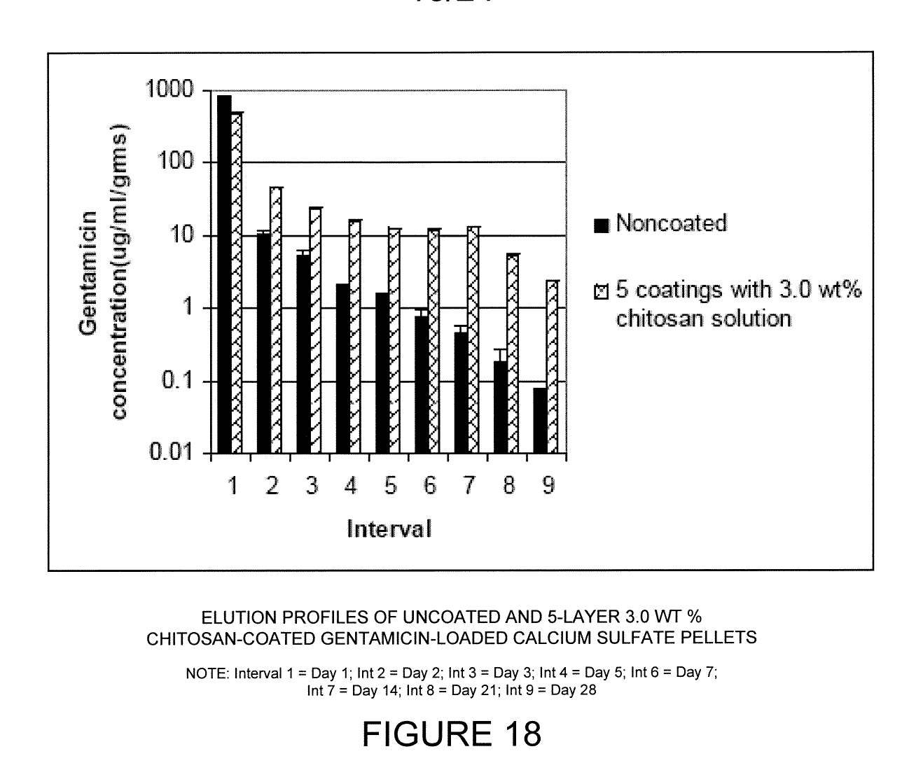

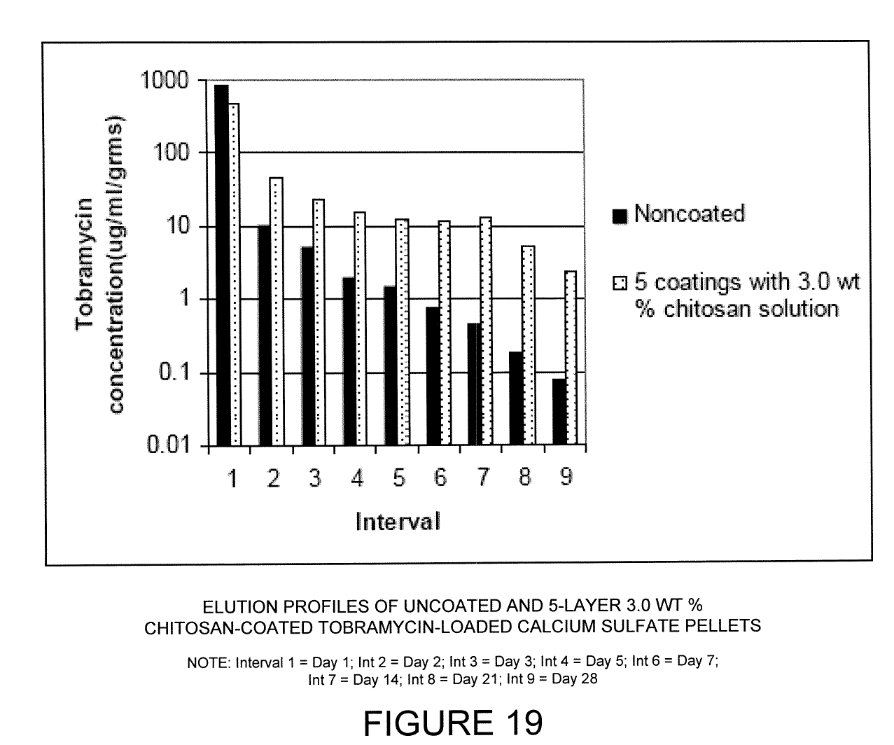

FIG. 18 shows the elution profiles for two variations of gentamicin-loaded calcium sulfate pellets (uncoated vs. five-layer chitosan coated with 3.0 wt %). Time intervals are not linear. Peak concentrations of 852 to 490 μg/ml/g occurred on day 1. Elution rates remained significantly higher with passing time for the coated pellet. The elution profiles for corresponding tobramycin-loaded calcium sulfate pellets are shown in FIG. 19.

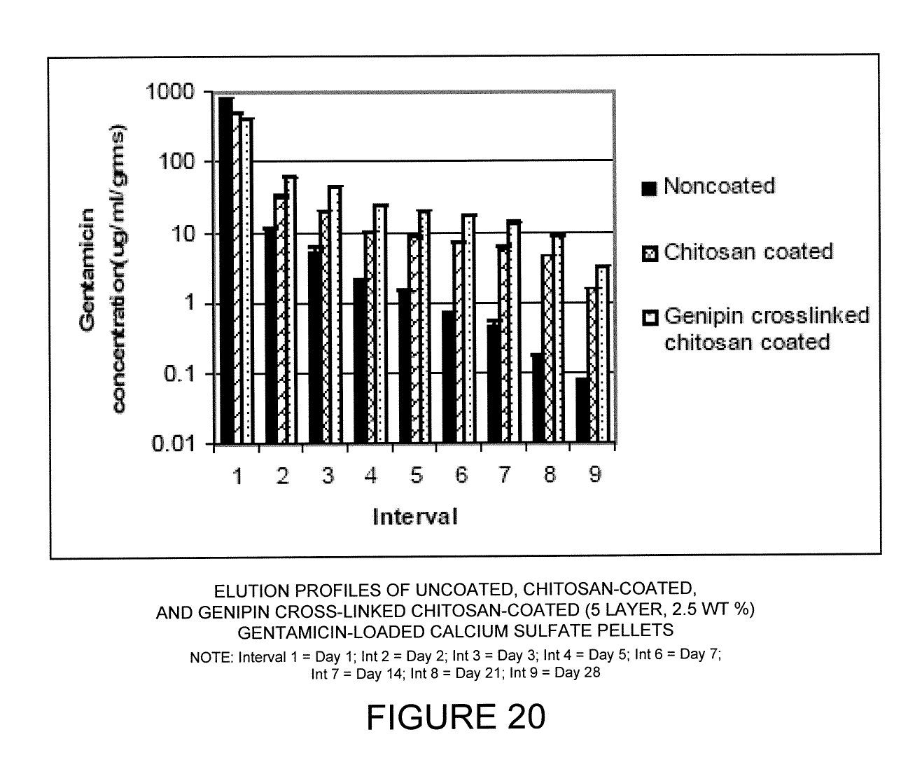

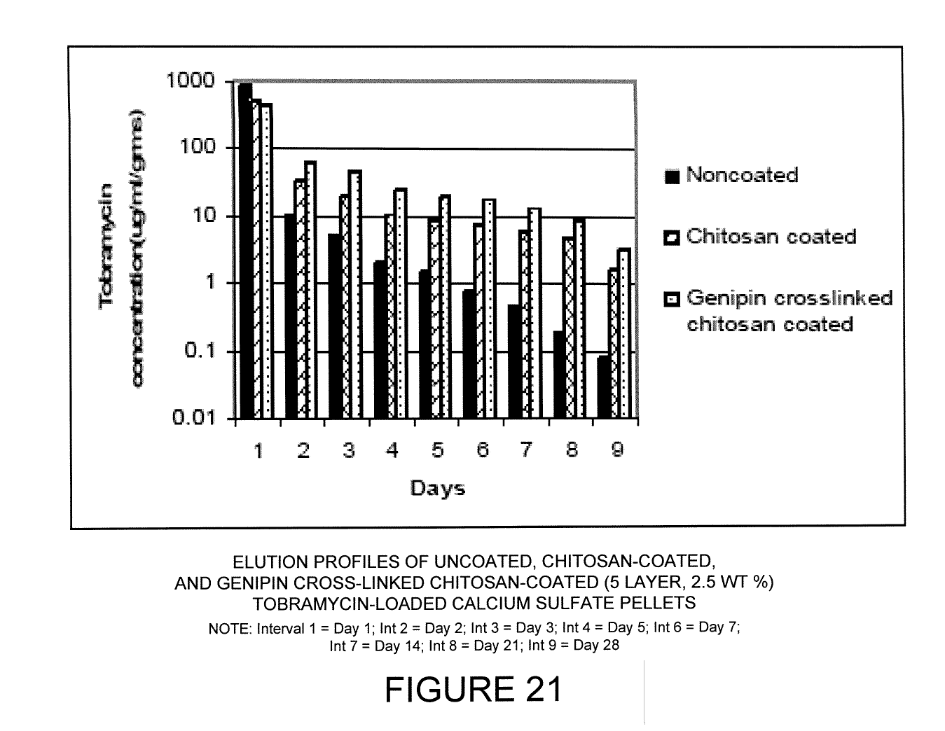

FIG. 20 demonstrates the effect of cross-linking the chitosan coating (2.5 wt %). It shows the dissolution profiles for three variations of gentamicin-loaded calcium sulfate pellets (uncoated; plain chitosan coated; cross-linked chitosan coated). The chitosan coatings were 5 layers. Peak concentrations of 852 to 425 μg/ml/g occurred at day 1. The cross-linked pellets showed the highest elution rates for the later time periods. The elution profiles for corresponding tobramycin-loaded calcium sulfate pellets are shown in FIG. 21.

While 87.4% DDA chitosan was used for several of the experiments described herein, other DDA percentages may be used.

In general, as the number of coatings on the calcium sulfate pellets increased, the dissolution and elution profile improved with time. Similarly, the cross-linked chitosan coating produced a more uniform coating with a reduction in dissolution. A cross-linked chitosan coated pellet (2.5 wt % chitosan solution cross-linked 2.5 wt % with genipin) showed an increase in the elution profile by 16.0% and a decrease in the dissolution profile by 25.0%. A coating obtained from the genipin cross linking of the 2.5 wt %, 87.4% DDA chitosan solution was very stable, easy to coat, uniform, and had a slow degradation rate which helped in obtaining an extended release profile for 28 days. These results showed an improvement in the profiles when compared with the unlinked coating. The improvement in the chitosan coated pellets in comparison to previous studies was primarily in reduction in the initial burst effect during the first 24 hours.

The combination of calcium sulfate chitosan pellets and genipin cross-linked chitosan coated calcium sulfate pellets also decreased the degradation/dissolution rate and potentially lowered the drainage issue associated with plain antibiotic loaded calcium sulfate pellets.

The slow degradation of multiple chitosan coatings on calcium sulfate pellets extended the release times of antibiotics. This elution improvement was enhanced by genipin cross linking with multiple coatings. These effects can aid in the bone regeneration of infected and contaminated bone defects. Further, the use of optimized multiple cross linked chitosan coatings can improve localized delivery of antibiotics with calcium sulfate pellets. Also, the eluents released from the genipin cross linked antibiotics loaded calcium sulfate pellets, were found active against the bacteria in the zone of inhibition testing.

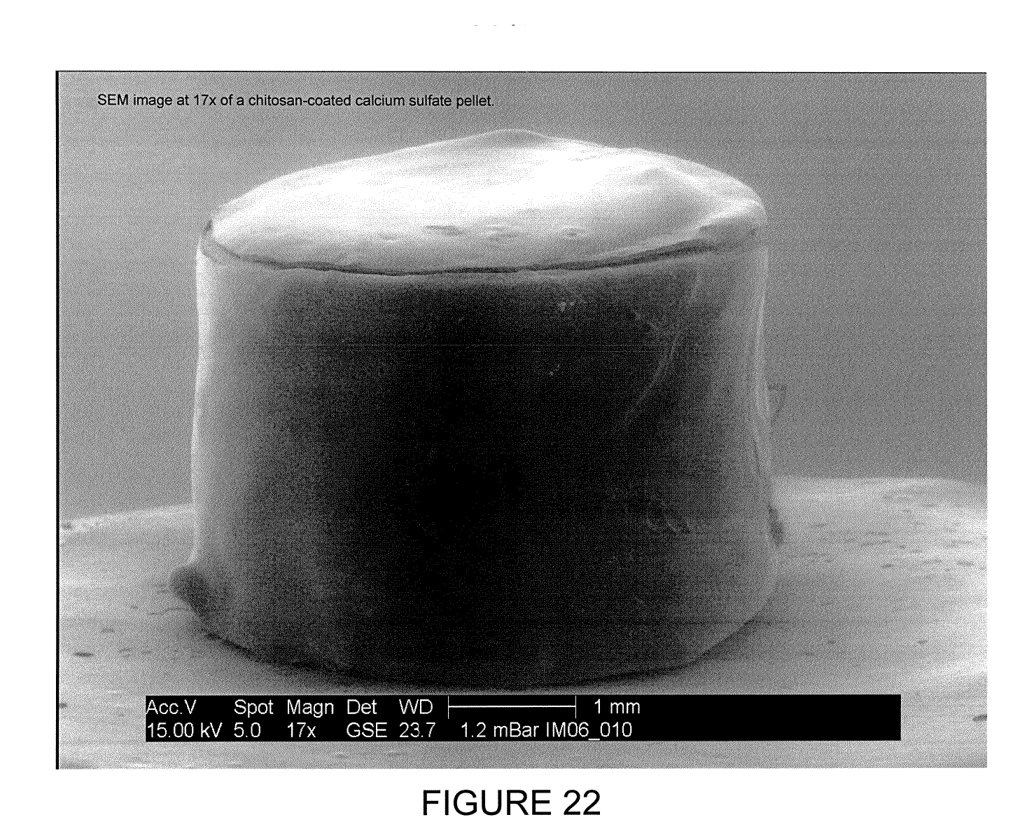

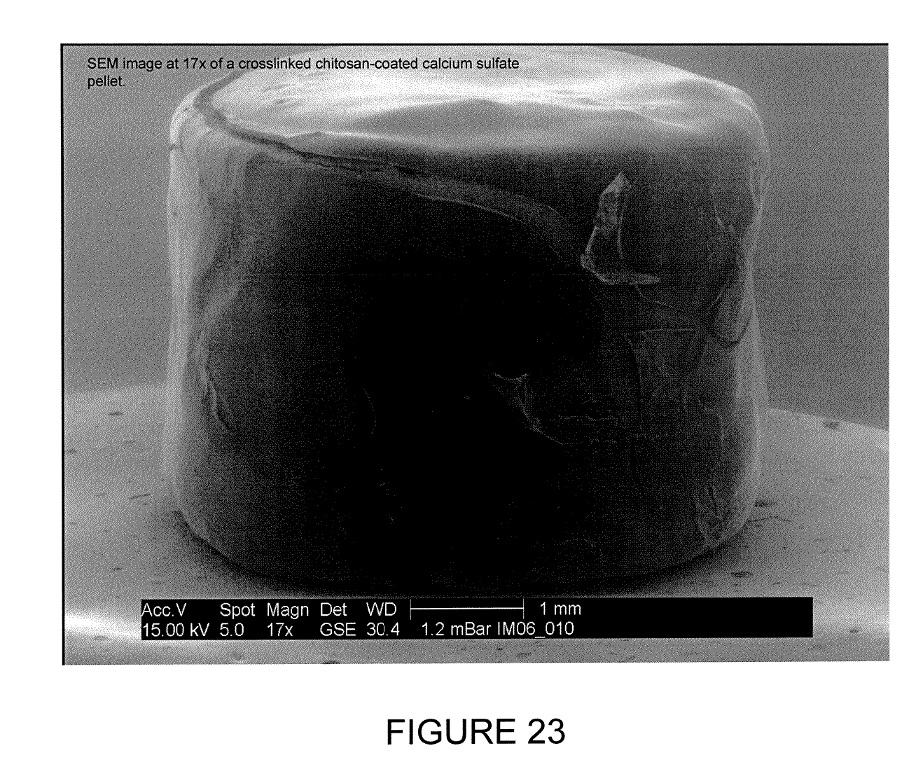

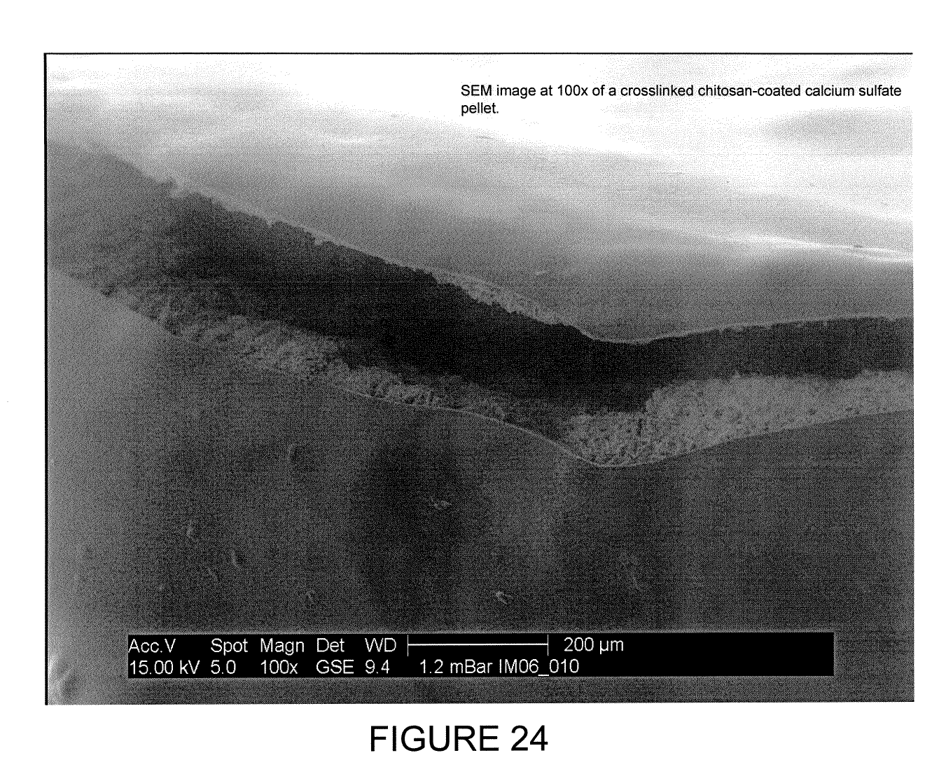

Some shapes may be more likely to cause cracking in the chitosan coating, which can create a pathway for leaching of the medicament and thus faster elution rates. As seen in FIG. 22-24, microscopic examination via SEM of the surface morphology of several chitosan-coated pellets in cylindrical form shows cracking along the edges of the pellet. Alternative shapes, such as spherical beads, may be used to prevent this effect and resultant leaching. Beads have no edges and therefore allow for a smooth coating surface. The cross-linked chitosan layer on spherical beads has remained intact after six hours and four days.

The opportunity to use these calcium sulfate vehicles also applies to the emerging field of growth factors and proteins. By the same mechanisms previously stated, growth factors and proteins can be delivered to a localized site and be released at a more desirable rate and for a longer duration than the current methodologies available at the present. In addition, therapeutic agents (growth factors, drugs, antibiotics, other medicants) may be added to initial solutions to make composite microspheres containing therapeutic agents. This will allow those compounds to be released at a slower, more controlled rate that will maintain a high local concentration of the therapeutic agent for an extended period of time.

Another advantage of the present invention is that the material may formulated as spheres or microspheres that can be fused together to form complex shapes. This would allow custom grafts and applications to be designed to fit to be applied to any site. The present invention may also be used in conjunction with a bone grafting or replacement material.

Thus, it should be understood that the embodiments and examples have been chosen and described in order to best illustrate the principles of the invention and its practical applications to thereby enable one of ordinary skill in the art to best utilize the invention in various embodiments and with various modifications as are suited for the particular uses contemplated. Even though specific embodiments of this invention have been described, they are not to be taken as exhaustive. There are several variations that will be apparent to those skilled in the art. Accordingly, it is intended that the scope of the invention be defined by the claims appended hereto.

We claim:

1. A medicament delivery system, comprising:
one or more delivery vehicles, said vehicle comprising a core coated at least in part with chitosan, said core comprising a combination of calcium sulfate and one or more medicaments.

2. The system of claim 1, wherein a plurality of said delivery vehicles are placed at the site where the medicament is to be delivered.

3. The system of claim 1, wherein one or more of the medicaments is an antibiotic.

4. The system of claim 3, wherein the antibiotic is one or more of the following: tobramycin, gentamicin, or daptomycin.

5. The system of claim 1, wherein one or more of the medicaments is an anesthetic.

6. The system of claim 5, wherein the anesthetic comprises lidocaine.

7. The system of claim 1, wherein one or more of the medicaments comprises a growth factor or protein.

8. The system of claim 1, wherein the core is completely encased in the chitosan coating.

9. The system of claim 1, wherein the chitosan coating comprises one or more chitosan layers.

10. The system of claim 9, wherein the chitosan coating comprises five layers of chitosan.

11. The system of claim 1, wherein the chitosan coating is cross-linked with a cross-linking agent.

12. The system of claim 11, wherein the cross-linking agent is genipin.

13. The system of claim 1, wherein the vehicles are bead-shaped or pellet-shaped.

14. A method for making a medicament delivery vehicle comprising a calcium sulfate core, comprising the steps of:
mixing calcium sulfate with one or more medicaments to create a vehicle core; and
coating the core with a layer of chitosan.

15. The method of claim 14, wherein the step of coating is repeated so there are multiple layers of chitosan in the chitosan coating.

16. The method of claim 14, further comprising the step of cross-linking the chitosan layer with a cross-linking agent.

17. The method of claim 16, wherein the cross-linking agent is genipin.

18. The method of claim 14, wherein the step of coating comprises immersing the vehicle core in a solution containing from 1% to 3.5% by weight chitosan.
